(19) United States
(12) Patent Application Publication
(10) Pub. No.: US 2011/0306087 A1
(43) Pub. Date: Dec. 15, 2011

(54) APPARATUS TO ANALYZE A BIOLOGICAL SAMPLE

(75) Inventors: Paolo Galiano, Padova (IT); Alessandro Mansutti, Gemona del Friuli (IT)

(73) Assignee: ALIFAX HOLDING SPA, Polverara (IT)

(21) Appl. No.: 13/203,200

(22) PCT Filed: Feb. 24, 2010
(86) PCT No.: PCT/IB2010/000369

§ 371 Date: Aug. 24, 2011

(30) Foreign Application Priority Data
Feb. 25, 2009 (IT) UD2009A000047

(51) Int. Cl. C12Q 1/04; C12M 1/34

(52) U.S. Cl. 435/34; 435/288.7

(57) ABSTRACT

An apparatus to analyze a biological sample contained in a container, comprising an examining device able to perform an optical measurement on the biological sample, based on light-scattering technology, at least of the bacterial growth in the biological sample. The examining device is provided with emitter means able to emit a first light beam toward the container of the biological sample and sensor means able to detect at least a light beam diffused from the container of the biological sample and to transmit a relative signal, correlated to the light beam diffused, to a control unit which is able to process, directly or indirectly, the signal in order to verify at least the possible bacterial growth in the biological sample. The container is disposed along a determinate lying plane and comprises at least a containing micro-element able to contain at least a part of the biological sample and inside which a liquid culture ground is provided so as to allow bacterial growth in the biological sample. The emitter means and sensor means are able to be located on each occasion in correspondence with the containing micro-element. The sensor means comprise first sensor means disposed on the same side of the emitter means with respect to the lying plane, and second sensor means disposed on the opposite side of the emitter means with respect to the lying plane. The first sensor means are able to detect a back-scattering radiation coming from the determinate containing micro-element. The second sensor means are able to detect a forward-scattering radiation coming from the determinate containing micro-element, from which back-scattering radiation and forward-scattering radiation derive respective first and second signals transmitted to the control unit.

### FIELD OF THE INVENTION

The present invention concerns an apparatus to analyze a liquid or solid biological sample, in particular to measure the bacterial cultural growth in a liquid culture ground, with the purpose of making a bacteriological and diagnostic search in general, or to measure turbidity for the purposes of a McFarland standardization for a sensitivity test, so as to make an analysis for sensitivity to antibiotics, or antibiogram, of a clinical or normal type. The biological sample to be analyzed can be for example urine, bronchial aspirate, blood, diluted or lysed blood or other.

### BACKGROUND OF THE INVENTION

An analysis apparatus is known from document WO-A-2006/021519 in the name of the present Applicant, to detect the presence and possibly identify the bacteria in a biological sample, such as blood or urine.

In the known apparatus, based on light-scattering, a laser light emitter is provided which strikes a laser beam on a test tube or flacon made of transparent glass or plastic containing a culture liquid inoculated with the biological sample. The light diffused by the bacteria in suspension during growth is detected by suitable sensors which transmit the relative signals to a processing unit which determines the presence of bacteria, and possibly classifies or identifies the bacteria possibly present.

In this type of apparatus, which has shown itself to be efficacious and advantageous, the need has emerged to make everything more compact and safe for the worker and for the work environment. This because the known apparatus, due to the use of analysis recipients or flacons, entails a big overall bulk.

This also leads to a high production of potentially infected material, which represents a risk for the worker and requires a complex disposal process?.

Furthermore, given the ever greater number of analyses required, the problem of productivity of such analysis apparatuses is a very urgent one.

Purpose of the present invention is to achieve an analysis apparatus that allows to reduce the operating bulk and the production of potentially infected material.

The Applicant has devised, tested and embodied the present invention to overcome the shortcomings of the state of the art and to obtain these and other purposes and advantages.

### SUMMARY OF THE. INVENTION

The present invention is set forth and characterized in the independent claims, while the dependent claims describe other characteristics of the invention or variants to the main inventive idea.

In accordance with the above purpose, an analysis apparatus according to the present invention is used to analyze a biological sample contained in a container, in particular but not only in order to perform a bacteriological search to measure the bacterial growth in order to verify the presence of bacteria, and possibly to classify and identify them.

The analysis apparatus according to the present invention comprises an examining device able to perform an optical measurement on the biological sample, based on light-scattering technology, at least of the bacterial growth in the biological sample. The examining device is provided with emitter means able to emit a first light beam toward the container of the biological sample and sensor means able to detect at least a light beam diffused from the container of the biological sample and to transmit a relative signal, correlated to said light beam diffused, to a control unit which is able to process said signal, directly or indirectly, so as to verify at least the bacterial growth, if any, in the biological sample.

According to the present invention, the container is disposed along a determinate lying plane and comprises at least a containing micro-element able to contain at least a part of the biological sample and inside which a liquid culture ground is provided so as to allow bacterial growth in the biological sample.

By containing micro-element, here and hereafter in the description, we mean a well or micro-well of a plate or micro-plate commonly used in microbiology, a reading cell or micro-cell, also known as cuvette or micro-cuvette, or in any case a container with very limited sizes with respect to the flacons or test tubes that are traditionally used, for example between 3 mm and 20 mm in height and between 3 mm and 20 mm in nominal diameter.

According to the present invention, the emitter means and sensor means can be located on each occasion in correspondence with the containing micro-element.

The sensor means comprise first sensor means disposed on the same side of the emitter means with respect to said lying plane and second sensor means disposed on the opposite side to the emitter means, always with respect to the lying plane of the container.

The first sensor means are able to detect a back-scattering radiation coming from the determinate containing micro-element analyzed on each occasion, while the second sensor means are able to detect a forward-scattering radiation, always coming from the containing micro-element.

According to the present invention, from said back-scattering and forward-scattering radiations respective first and second signals are derived, transmitted to the control unit. The signals are used to verify the bacterial growth and all the necessary analyses. According to one embodiment, the emitter means are located so that the first light beam strikes the determinate containing micro-element from above or below.

According to a variant, the emitter means are located vertical with respect to the lying plane of the container, so that the relative first light beam strikes the containing micro-element substantially perpendicularly.

Advantageously, the particular combination of the innovative characteristics of the present invention allows an accurate analysis of the sample using light-scattering technology, irrespective of the sizes or shape of the container of the sample, because the emitter means and sensors operate using a ray of light that is directed vertically.

According to another variant, the emitter means are located inclined with respect to the lying plane of the container so that the relative first light beam strikes the containing micro-element with a desired inclination by an angle other than 90° with respect to a vertical reference direction. According to an advantageous embodiment, the container is a micro-plate of the type commonly used in microbiology (for example 96 or 384 wells) that develops along the lying plane, and said containing micro-elements are relative wells disposed in rows and columns along the lying plane.

Typically each of the micro-elements or wells has an upper aperture for the passage of the first light beam and the back-scattering radiation, and a bottom wall made of suitable material to allow the passage of the forward-scattering radiation.

Advantageously each of the wells has a lateral wall which is obscured to prevent the lateral diffusion of the light so as not to interfere with the other adjacent wells.

According to one embodiment the plates or micro-plates are made of polystyrene or methacrylate.

Advantageously, the first sensors and the second sensors are positioned, with respect to a determinate vertical direction, each at a defined angle comprised between about −90° and +90°.

It comes within the field of the present invention to use an examining device based on light-scattering technology to detect a back-scattering radiation coming from one side of a containing micro-element containing a biological sample in suspension in a liquid culture ground and a forward-scattering radiation from an opposite side of said containing micro-element, to measure at least the bacterial growth of the biological sample contained in the containing micro-element.

Advantageously, by said examining device of the light-scattering type, the growth curves of the bacteria possibly present in the sample analyzed are detected on one or more wells of the plate.

This detection, depending on the step of the analysis when it is made, can be used to determine the presence of bacteria (quick culture test), to identify the bacteria, or for the Raa test (residual antibiotic activity), for the clinical antibiogram test or other tests and analyses that require this type of detection. If the present invention is applied to analyze lysed blood, the apparatus operates with light beams perpendicular to the transparent micro-well, to prevent possible interference from the lysed globules.

On the contrary, in applications to transparent liquids, where the optical travel by parallel or vertical reading assumes greater accuracy, it is advantageous to use micro-wells or micro-cuvettes with an obscured lateral wall.

One advantage of using micro-plates or other containing micro-elements combined with light-scattering detection, compared with containers of the type normally used for light-scattering, is that the micro-plate can be suitably filled with the volumes of samples necessary, including reagents and various liquids for the detection, including fluorescent components, preventing any waste of material.

Furthermore, by using micro-plates or other similar containers of limited size, such as micro-arrays, micro-cuvettes or other according to the present invention, the bulk of the analysis apparatus and the production of potentially infected material is reduced.

Moreover, the productivity of a single analysis apparatus of the type described above is considerably increased, as it can operate in quick succession on a plurality of adjacent containing micro-elements, each with a precise and sufficient, not excessive, quantity of sample and culture liquid, thus limiting wastes of material and analysis time.

The present invention also comprises a method to analyze a biological sample contained in a container disposed along a determinate lying plane and comprising at least a containing micro-element able to contain at least part of the biological sample and inside which a liquid culture ground is provided to allow bacterial growth in the biological sample. The method provides an examination step in which an optical measurement is carried out, based on light-scattering technology, at least of the bacterial growth in the biological sample, by emitting a first light beam toward the biological sample, detecting at least a light beam diffused from the container of the biological sample and by processing, directly or indirectly, signals correlated to said light beam diffused. According to the present invention, the method provides to detect, on the same side with respect to the lying plane associated with the containing micro-element from which said first light beam is emitted, a back-scattering radiation coming from the determinate containing micro-element and, on the side opposite the lying plane associated with the containing micro-element, a forward-scattering radiation coming from the determinate containing micro-element. The method also provides to derive said signals from the back-scattering and forward-scattering radiations.

### BRIEF DESCRIPTION OF THE DRAWINGS

These and other characteristics of the present invention will become apparent from the following description of a preferential form of embodiment, given as a non-restrictive example with reference to the attached drawings wherein:

FIG. 1 is a schematic representation of a container used with the present invention;

FIG. 2 is a schematic representation of part of the apparatus according to the present invention;

FIG. 3 is an enlarged detail of FIG. 2;

FIG. 4 is a variant of a part of the apparatus in FIG. 2;

FIG. 5 is a schematic representation of an embodiment of the apparatus according to the present invention.

### DETAILED DESCRIPTION OF A PREFERENTIAL FORM OF EMBODIMENT

With reference to the attached drawings, an apparatus 10 according to the invention is used to analyze a biological sample 11, in particular to measure the bacterial growth and to identify the presence of bacteria contained in the biological sample. The apparatus 10 applies the light-scattering technique to small size containers, such as for example micro-plates (96 wells) or micro-arrays (384 wells), micro-cuvettes or glass micro-cells, micro-cuvettes with the lateral wall obscured (to prevent the diffusion of the light beam).

The same apparatus 10 can be used to measure turbidity for the purposes of McFarland standardization for a sensitivity test, to carry out an analysis of sensitivity to antibiotics, or antibiogram, of the clinical or normal type.

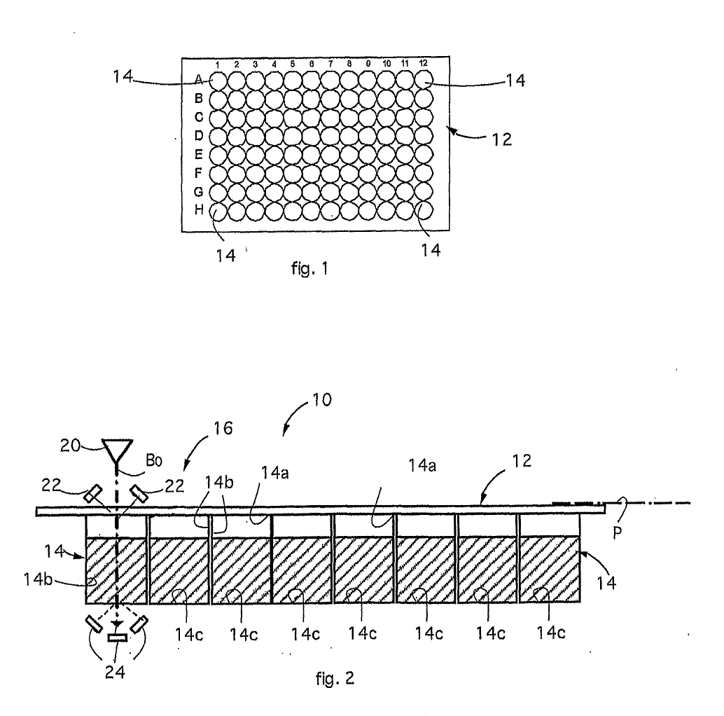

In this case, the apparatus 10 comprises a micro-plate 12, formed by 96 containing micro-elements or wells 14 (FIG. 1), disposed along a substantially horizontal lying plane P (FIG. 2).

The wells 14 function as containers for bacterial growth in a suitable liquid culture ground, for example eugenic medium, and for the possible biochemical reactions used to recognize the presence and/or identify the type of bacteria in the biological sample 11 to be analyzed.

The plates 12 can be suitably thermostated, for example at a temperature comprised between about 35° C. and 37° C., to promote the bacterial growth, and preferentially subjected to stirring.

For use with light-scattering technology, as required by the present invention, the plates 12 and the wells 14 are compatible with the desired electromagnetic radiation, preferably transparent or permeable to visible light.

The plates 12 employed, therefore, preferably have good optical qualities, typically with great clarity and uniformity, so that they have a high capacity of letting the light pass through the wells 14 in the analysis. For example, the plates 12 are made of polystyrene or methacrylate, which embody the above optimum optical properties.

Each well 14 has an upper aperture 14a, for the sample to be inserted and for the light to pass, and a bottom wall 14c, also permeable to light.

In the specific solution, where there are adjacent wells 14 in large quantities, their lateral walls 14b are blackened, or in any case rendered not transparent to light, so as to eliminate possible optical interferences of one well 14 with the adjacent wells. Typically, before inoculation, the biological sample is preserved in a test tube and a part of it is picked up, advantageously by means of a movement and selection unit, not shown in the drawings, and deposited in one or more of the wells 14 of the plate 12, to perform the desired analyses.

The apparatus 10 also comprises an examining device 16 based on light-scattering technology, by means of which a step of detection is carried out on one or more of the wells 14, in which suitable signals are generated by means of which the bacterial growth or inhibition kinetics in each well 14 are assessed, as illustrated hereafter in the description (FIGS. 2, 3 and 5).

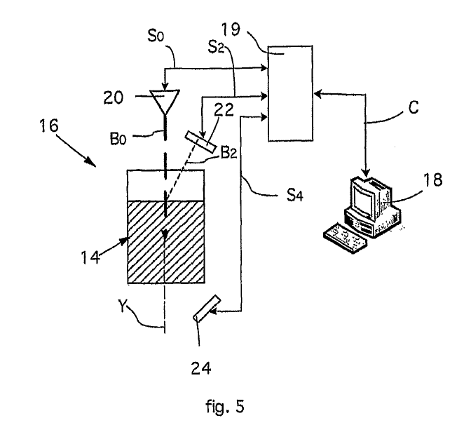

The apparatus 10 also comprises a control unit 18, for example an electronic calculator, in whose memory a computer program is loaded to manage the examining device 16. The control unit 18 is shown for convenience only in FIG. 5. The control unit 18 is associated, by means of a hardware connection C, to an interface card 19 which is able to control the activation/de-activation of the examining device 16 (FIG. 5), as shown schematically by the command and control signal S0.

The data collected by the examining device 16 are sent to the control unit 18 by the interface card 19, which amplifies, filters and processes the data collected.

In this way, the control unit 18 is able to evaluate the bacterial growth or inhibition kinetics in each well 14 (bacterial growth test).

The control unit 18 can also activate the sample movement and selection unit, if there is one.

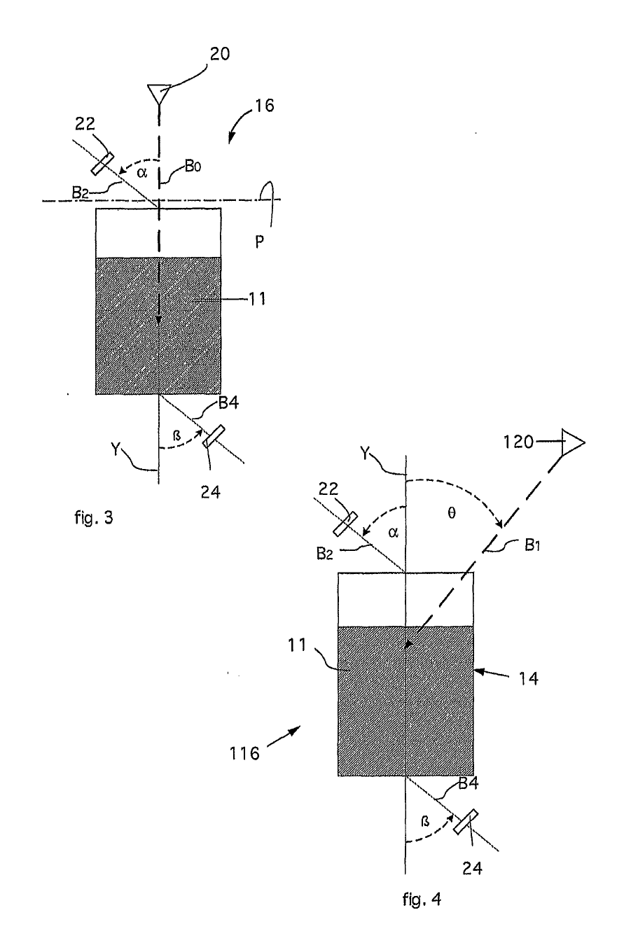

According to the embodiment shown in FIGS. 2 and 3, the examining device 16 comprises a light emitter 20, disposed vertically above or below the micro-plate 12 with respect to said plane P, in correspondence with the various wells 14 which make up the micro-plate 12; the emitter 20 is able to emit a laser light beam, polarized and collimated, indicated by B0, toward the well 14 to be analyzed, with an inclination of 90° with respect to the lying plane P of the micro-plate 12, which is an advantageous solution in the analysis of lysed blood so as to prevent possible interferences of the lysed globules. The light beam B0 is diffused from the relative well 14 of the micro-plate 12, in particular by the biological sample growing in the liquid culture ground inside the well 14 of the micro-plate 12.

A plurality of sensors 22, 24 are associated with the emitter 20, and are located vertically above and below the lying plane P of the micro-plate 12.

In particular, according to the embodiment shown in FIG. 2, two first sensors 22 are provided, located on the same side of the emitter 20 with respect to the micro-plate 12, and three second sensors 24, located on the opposite side of the emitter with respect to the lying plane P of the micro-plate 12. It is understood that the number of sensors can be different from this.

Both the first 22 and the second 24 sensors are positioned at different angles with respect to the vertical direction, indicated by Y and coincident with the path of the light beam B0. The angles are indicated by α and β in FIG. 3 and are typically comprised between −90° and +90° with respect to the vertical Y. In the solution given as an example in FIG. 2, there are two upper sensors 22 disposed respectively at about +45° and −45° and three lower sensors, of which two external disposed respectively at about +45° and −45°, and a central one disposed at 0°, that is, aligned with the vertical Y.

In a different form of embodiment, not shown, the first and second sensors 22, 24 can be positioned at different angles, for example at about 0°, 30° and 90°.

Furthermore, the sensors 22, 24 are positioned at a variable distance from the relative well 14, according to the requirements of the analysis. In fact, once struck by the light beam B0 emitted, the biological sample 11 in the well 14, with the presence of duplicating bacteria, emits beams of diffused light B2, B4 which the control unit 18 processes so as to supply specific curves that express the development of the bacterial growth over time.

The sensors 22 and 24 periodically detect the beams B2, B4 of diffused light emitted by the biological sample 11 contained in the well 14 and transmit them as signals S2, S4 to the control unit 18.

In particular, the first sensors 22 periodically detect the back-scattering, indicated by the light beam B2, of the light striking the well 14, whereas the second sensors 24 detect the forward-scattering, indicated by the light beam B4, of the light through the well 14.

FIG. 3 is a detail referring to a single well 14, in which a single first sensor 22 and a single second sensor 24 are used, disposed on opposite sides with respect to the lying plane P of the micro-plate 12.

The sensors 22 and 24 transmit the relative signals S2, S4, correlated to the light beams B2, B4 detected, to the interface card 19, which converts them from analog to digital, transmitting the relative value of forward-scattering and back-scattering to the control unit 18 which processes the signals for the required analyses and'evaluations.

Once converted, the signals S2, S4 allow to determine a first and a second curve, respectively associated with each of the sensors 22, 24, of the development over time of the turbidity of the bacterial suspension, that is, of the bacterial growth. The control unit 18 processes the signals S2, S4 so as to determine, from said curves, two corresponding differential curves.

Each differential curve is given by the difference respectively between the first curve and a first instantaneous value of turbidity obtained at the start of the detection, detected by the first sensor 22, and between the second curve and a second instantaneous value of turbidity at the start of the detection, detected in correspondence with the second sensor 24.

The control unit 18 advantageously comprises memorization means in which classification data are memorized, by means of which, from the development of the two differential curves, the type of bacterium present in the biological sample where the bacterial growth takes place, or the family it belongs to, is found.

The first curve derived from the signal obtained by the first sensor 22 relates to the presence of bacteria and consequent measurement of the bacterial load over time. The second curve derived from the signal obtained by the second sensor 24 on the contrary is more characterized by the morphology of the bacteria.

Starting from the differential curves, specific mathematical parameters are extrapolated for each curve, based on non-linear regression models. It is also possible to combine the homologous parameters of the two curves (for example by calculating ratios, or differences or sums) so as to obtain further derived parameters.

The totality of these parameters provides a synthetic description of the characteristics of the curves of the bacterium present in the sample examined.

FIG. 4 shows a variant embodiment of the examining device 16, indicated for convenience by the reference number 116, where parts identical to those shown in the embodiment in FIGS. 1-3 are identified by the same reference numbers. The examining device 116 provides an emitter 120 that is not located perpendicular with respect to the micro-plate but is inclined by a determinate angle θ with respect to the vertical Y. The positions of the sensors 22 and 24 remain as described in the various possibilities in FIGS. 1-3. In this way, at least part of the light-scattering components generated by a final segment of the volume of illuminated sample, and directed at 90° with respect to the direction of the radiation emitted B1, passes through the bottom wall 14c of the well 14 to be detected by the sensors 24, instead of being absorbed by the lateral walls 14b, as in the case of an emitter 120 located perpendicular with respect to the micro-plate.

1-11. (canceled)

12. An apparatus to analyze a biological sample contained in a container, comprising an examining device able to perform an optical measurement on the biological sample, based on light-scattering technology, at least of the bacterial growth in the biological sample and provided with emitter means able to emit a first light beam toward the container of the biological sample and sensor means able to detect at least a light beam diffused from the container of the biological sample and to transmit a relative signal, correlated to said light beam diffused, to a control unit which is able to process, directly or indirectly, said signal, in order to verify at least the possible bacterial growth in the biological sample, wherein said container is disposed along a determinate lying plane and comprises at least a containing micro-element able to contain at least a part of the biological sample and inside which a liquid culture ground is provided so as to allow bacterial growth in the biological sample, said emitter means and sensor means being able to be located on each occasion in correspondence with the containing micro-element, said sensor means comprising first sensor means disposed on the same side of the emitter means with respect to said lying plane, and second sensor means disposed on the opposite side of the emitter means) with respect to said lying plane, said first sensor means being able to detect a back-scattering radiation coming from the determinate containing micro-element and said second sensor means being able to detect a forward-scattering radiation coming from the determinate containing micro-element, from which back-scattering radiation and forward-scattering radiation derive respective first and second signals transmitted to the control unit.

13. The apparatus as in claim 12, wherein said emitter means are placed so that the first light beam strikes, from above or from below, on the determinate containing micro-element.

14. The apparatus as in claim 12, wherein said emitter means are placed vertically with respect to the lying plane of said container, so that the relative first light beam strikes substantially perpendicularly on said containing micro-element.

15. The apparatus as in claim 12, wherein said emitter means are placed inclined with respect to the lying plane of said container so that the relative first light beam strikes on said containing micro-element with a desired inclination of an angle different from 90° with respect to a vertical direction.

16. The apparatus as in claim 12, wherein said container is a micro-plate which develops along said lying plane and said micro-containing elements are relative wells disposed in rows and columns along said lying plane.

17. The apparatus as in claim 16, wherein each of the wells has an upper opening for the passage of the first light beam and of the back-scattering radiation and a bottom wall made of material suitable to allow the passage of the forward-scattering radiation.

18. The apparatus as in claim 16, wherein each of the wells has a lateral wall, which is obscured so as to impede the lateral diffusion of light in order not to interfere with the other adjacent wells.

19. The apparatus as in claim 12, wherein the plates or micro-plates are made of polystyrene or methacrylate.

20. The apparatus as in claim 12, wherein the first sensors means and the second sensors means are positioned, with respect to a determined vertical direction, each at a defined angle comprised between about −90° and +90°.

21. A method to detect a back-scattering radiation coming from one side of a containing micro-element containing a biological sample in suspension in a liquid culture ground and a forward-scattering radiation from the opposite side of said containing micro-element so as to measure at least the bacterial growth of the biological sample contained in the containing micro-element, using an examining device based on light-scattering technology in an apparatus as in claim 12.

22. A method to analyze a biological sample contained in a container, disposed along a determinate lying plane and comprising at least a containing micro-element able to contain at least a part of the biological sample and inside which a liquid culture ground is provided so as to allow bacterial growth in the biological sample, said method providing an examination step in which an optical measurement is made, based on light-scattering technology, at least of the bacterial growth in the biological sample by emitting a first light beam toward the container of the biological sample, by detecting at least a light beam diffused from the container of the biological sample and by processing, directly or indirectly, signals correlated to said light beam diffused, the method providing to detect, on the same side with respect to the lying plane associated with the containing micro-element from which said first light beam is emitted, a back-scattering radiation coming from the determinate containing micro-element and, from the opposite side with respect to the lying plane associated with the containing micro-element, a forward-scattering radiation coming from the determinate containing micro-element, deriving said signals from said back-scattering radiations and forward-scattering radiations
